(19) United States
(12) Patent Application Publication
(10) Pub. No.: US 2021/0137824 A1
(43) Pub. Date: May 13, 2021

(54) METHOD FOR PRODUCING COSMETIC COMPOSITION COMPRISING NATURAL PLANT AND COSMETIC COMPOSITION PRODUCED THEREBY

(71) Applicant: RAFIQ COSMETICS CO. LTD., Cheonan-si, Chungcheongnam-do (KR)

(72) Inventor: Pomjoo LEE, Yougin-si, Gyeonggi-do (KR)

(73) Assignee: RAFIQ COSMETICS CO. LTD., Cheonan-si, Chungcheongnam-do (KR)

(21) Appl. No.: 16/766,966

(22) PCT Filed: Nov. 27, 2018
(86) PCT No.: PCT/KR2018/014682

§ 371 Date: Nov. 11, 2020

(30) Foreign Application Priority Data
Nov. 30, 2017 (KR) 10-2017-0163820
Nov. 27, 2018 (KR) 10-2018-0148069

(51) Int. Cl. A61K 8/9789; A61K 8/362; A61K 8/365; A61K 8/23; A61Q 19/00

(52) U.S. Cl. CPC A61K 8/9789; A61K 8/362; A61K 8/365; A61K 2800/10; A61Q 19/00; A61K 2800/805; A61K 8/23

(57) ABSTRACT

The present disclosure relates to a method for preparing a cosmetic composition comprising a natural plant and a cosmetic composition prepared thereby. The cosmetic composition preparation method comprises the steps of preparing a natural plant, impregnating the natural plant with an aqueous solution comprising a base or acid and purified water, and softening the aqueous solution while heating the aqueous solution.

### TECHNICAL FIELD

The present disclosure relates to a method for preparing a cosmetic composition comprising a natural plant and a cosmetic composition prepared thereby.

### BACKGROUND ART

Cosmetic products, as products of cleaning and beautifying the human body, are sensitive to fashion or trends in related industries. With the economy developing and consumption rising recently, it is a trend to subdivide cosmetic products into more product groups and to select and use raw materials which are safer and have higher efficacies as raw materials used. Although there are original functions such as moisturizing power, elasticity and others which should be basically possessed by cosmetic products, it has recently been required that cosmetic products should have incidental functions such as fragrance, usability, properties and others as well as basic efficacies of cosmetic products.

Further, it is a trend taken into the mainstream of the industry to find material which is safer to the human body and is nature-friendly and unknown natural material as material or raw material that has recently been used in cosmetic products, and such a trend is stronger particularly in plant materials. Research on the plant materials has been continued to find an organic plant material, a pesticide-free plant material, and materials of areas which are safer and beyond one's reach such as Amazon, Africa, the Antarctic, the ocean floor and others.

Meanwhile, there is a case of using, as material for cosmetic products, an extract obtained by extracting active ingredients of plants in line with recent trends. Since most of the active ingredients is lost in a process of extracting the active ingredients of the plants, there is a problem that the active ingredients of the plants have very low effects.

Therefore, it has recently been required to conduct a research of maximally maintaining shapes of the plants while using materials of plants and the like, and enabling the active ingredients of the plants to be maximally used.

### DISCLOSURE

### Technical Problem

In order to solve such a problem, an objective of the present disclosure to provide a cosmetic composition preparation method which enables an active ingredient of the natural plant to be maximally used while maintaining shape of a natural plant.

Furthermore, the other objective of the present disclosure to provide a cosmetic composition prepared by the cosmetic composition preparation method.

The objectives of the present disclosure are not limited to the above-described contents. The objectives of the present disclosure can be understood from the entire contents of the present specification, and those skilled in the art to which the present disclosure pertains will not have any difficulties in understanding additional objectives of the present disclosure.

### Technical Solution

A cosmetic composition preparation method according to an embodiment of the present disclosure for achieving the above-mentioned objective comprises the steps of preparing a natural plant, impregnating the natural plant with an aqueous solution comprising a base or acid and purified water, and softening the aqueous solution while heating the aqueous solution.

In addition, the acid may include a carboxyl group or a functional group of sulfonic acid.

In addition, the acid may include an organic acid including at least one of citric acid, tartaric acid, lactic acid, glycolic acid, formic acid, malic acid, oxalic acid, salicylic acid, ascorbic acid, and glucono delta-lactone, or an inorganic acid including at least one of hydrochloric acid, sulfuric acid, nitric acid, phosphoric acid, and carbonic acid.

In addition, the acid may be in a concentration range of 0.1 to 4 wt % with respect to the total weight of the aqueous solution.

In addition, the base may include a functional group of hydroxyl group or amine group at an end thereof.

In addition, the base may include at least one of potassium hydroxide, ammonium hydroxide and sodium hydroxide, or at least one of triethanolamine, aminomethyl propanol, and tromethamine.

In addition, the softening step may comprise performing a process of softening the aqueous solution in a temperature range of 40 to 95° C. for a softening time of 30 to 240 minutes.

In addition, the softening step may comprise performing a process of softening the aqueous solution in a temperature range of 70 to 90° C. for a softening time of 60 to 180 minutes.

In addition, the softening step may comprise performing a process of softening the aqueous solution in a temperature range of more than 90° C. to not more than 95° C. for a softening time of 30 to 150 minutes.

In addition, the cosmetic composition preparation method may further comprise a step of drying the natural plant before the step of impregnating the natural plant with the aqueous solution.

In addition, the drying step may comprise performing a process of drying the natural plant such that the natural plant after performing the drying process has a moisture content range of 5 to 15% with respect to the total weight of a dried natural plant.

In addition, the step of preparing the natural plant may further comprise a step of pulverizing the natural plant to a size of 50 to 10,000 μm.

In addition, the cosmetic composition preparation method may further comprise a step of confirming whether the natural plant has been softened or not after the softening step, wherein the confirming step comprises repeatedly performing the softening step for a softening time range of 15 to 45 minutes when the softening process is less accomplished.

In addition, the cosmetic composition preparation method may further comprise a step of washing the natural plant softened by the softening step by using purified water.

In addition, the washing step may comprise repeatedly performing a washing process using the purified water about three to six times.

In addition, the washing step may be performed to maintain a pH value of purified water finishing the washing process in the washing step to a range of 2.5 to 4.5.

In addition, the natural plant may include rose, sunflower, chrysanthemum, calendula, cornflower, chamomile, morning glory, rose moss, garden balsam, cherry, green tea, hibiscus, peppermint, Asiatic pennywort, Portulaca oleracea, mugwort, Yerba Santa, Taraxacum platycarpum Dahlst., Plantago asiatica L., Huttuynia cordata, Aloe barbadensis, Opuntia humifusa, white willow, and cactusfruit.

A cosmetic composition according to an embodiment of the present disclosure for achieving the above-mentioned objective comprises softened natural plant and purified water, wherein the softened natural plant is prepared by the cosmetic composition preparation method.

In addition, the softened natural plant and purified water may be mixed at a weight ratio of 1:1 to 1:10.

### Advantageous Effects

A cosmetic composition preparation method according to the present disclosure and a cosmetic composition prepared thereby can soften a natural plant while enabling shape of a softened natural plant to be maintained as it is.

Further, a cosmetic composition preparation method according to the present disclosure enables the softened natural plant material to be contained in cosmetic products while maintaining shape of a softened natural plant material, and enables the user to the softened natural plant material to be decomposed by an appropriate force when a user uses cosmetic products. The cosmetic composition preparation method according to the present disclosure does not destroy most of active ingredients of natural plants in a process of producing cosmetic products, but enables the active ingredients of the natural plants to be dissolved into the skin of the user when the user uses the cosmetic products.

### DESCRIPTION OF DRAWINGS

FIG. 1 is a photograph of a cosmetic composition containing a softened natural plant prepared according to an embodiment of the present disclosure.

FIG. 2 is a photograph of a cosmetic product containing a softened natural plant prepared according to an embodiment of the present disclosure.

FIG. 3 is a photograph showing that a user uses a cosmetic product produced according to an embodiment of the present disclosure.

FIG. 4 is a photograph showing an effect of dissolving softened floral leaves on the skin of the user after a user uses a cosmetic product produced according to an embodiment of the present disclosure as in FIG. 3.

FIG. 5 to FIG. 16 are photographs of resulting products for Examples and Comparative Examples of the present disclosure.

### MODE FOR DISCLOSURE

Hereinafter, preferred embodiments of the present disclosure will be described. However, the embodiments of the present disclosure may be modified in many different forms and the scope of the present disclosure should not be construed as being limited to the embodiments set forth herein. Further, the embodiments of the present disclosure are provided in order to more fully describe the present disclosure to a person having ordinary skill in the art.

The present inventors have completed the present disclosure as a result of repeating a long research for finding a preparation method for allowing ingredients of the natural plant to be penetrated into the skin by dissolving the natural plant on epidermis of skin of the user only when a user uses the cosmetic product while maintaining shape of a natural plant within a cosmetic product.

A cosmetic composition preparation method according to an embodiment of the present disclosure comprises a step of preparing a natural plant, a step of impregnating the natural plant with an aqueous solution comprising a base or acid and purified water, and a step of softening the aqueous solution while heating the aqueous solution.

On the other hand, the cosmetic composition preparation method may further comprise a step of drying the natural plant before the step of impregnating the natural plant with the aqueous solution. For example, the drying step comprises performing a process of drying the natural plant such that the natural plant after performing the drying process may have a moisture content range of 5 to 15% with respect to the total weight of a dried natural plant. The natural plant may be softened more accurately within the range.

A cosmetic composition passing through a preparation process described above comprises a softened natural plant. The natural plant may be said to be material closest to material which is safer to the human body and nature-friendly compared to synthetic materials. Accordingly, when the natural plant itself is used as a cosmetic composition used in the production of a cosmetic product, a final product may sufficiently obtain an effect of material as above.

Further, the cosmetic composition prepared through the above-described process may have a visually excellent effect since the natural plant exists in a softened state, and shape of the natural plant is maintained. Further, in case of an existing preparation method, it is hard to examine shape of the natural plant as the natural plant is used as an extract although a natural plant is used as material for a cosmetic product, or the natural plant has been used in a finely pulverized state such that the shape of the natural plant cannot be recognized. In case of a preparation method according to the present disclosure, the natural plant maintains shape to an extent capable of performing a process of visually observing what kind of natural plant the shape of the natural plant is as the natural plant exists in a softened state. Therefore, since ingredients having efficacies which are good to the human body in the natural plant not only can be intactly stored in the softened natural plant, but also maintain shape within a cosmetic product, i.e., a final product to be described later, the ingredients have a very excellent visual effect. Namely, consumers can visually feel that the natural plant exists within an actual cosmetic product.

Meanwhile, although the natural plant may be any natural plants existing in nature, examples of the natural plant may include rose, sunflower, chrysanthemum, calendula, cornflower, chamomile, morning glory, rose moss, garden balsam, cherry, green tea, hibiscus, peppermint, Asiatic pennywort, Portulaca oleracea, mugwort, Yerba Santa, Taraxacum platycarpum Dahlst., Plantago asiatica L., Huttuynia cordata, Aloe barbadensis, Opuntia humifusa, white willow, and cactusfruit.

For example, since rose petals are very excellent in effects of maintaining elasticity of the skin and maintaining health of the person when the rose petals are used in the skin of a person by comprising large amounts of polyphenol and derivatives thereof having excellent antioxidant effects and comprising large amounts of essential oil ingredients. Further, the present disclosure not only has an excellent visual effect, but also can maximize utilization of the active ingredients since active ingredients as above exist in a cosmetic composition and a cosmetic product including the cosmetic composition in a state that the active ingredients maintain their shapes while most of the active ingredients are being preserved within the rose petals.

A cosmetic composition preparation method according to the present disclosure comprises a step of impregnating the natural plant with an aqueous solution comprising a base or acid and purified water. As a non-restrictive example, the natural plant may be impregnated such that the natural plant is completely soaked with the aqueous solution comprising the base or acid and purified water.

On the other hand, the acid may include a carboxyl group or a functional group of sulfonic acid. The acid may include an organic acid or inorganic acid. As a specific example, the acid may include an organic acid including at least one of citric acid, tartaric acid, lactic acid, glycolic acid, formic acid, malic acid, oxalic acid, salicylic acid, ascorbic acid, and glucono delta-lactone, or an inorganic acid including at least one of hydrochloric acid, sulfuric acid, nitric acid, phosphoric acid, and carbonic acid. However, the present disclosure is not limited thereto.

The acid can soften the natural plant by allowing a carboxyl group contained in the acid or an acidic functional group of sulfonic acid or the like to cut a portion of a bond between celluloses existing in a natural plant. Accordingly, the natural plant is softened while maintaining its shape, and the celluloses can be disintegrated only by a finger rubbing force when a user uses a cosmetic product. Eventually, the natural plant is completely disintegrated with little effort when the user uses the cosmetic product while maintaining its shape in a softened state, and most of active ingredients of the natural plant can be penetrated into the skin of the user.

The acid may be in a concentration range of 0.1 to 4 wt % with respect to the total weight of the aqueous solution. For example, the acid may be in a concentration range of 0.5 to 2 wt %. The natural plant can be appropriately softened in the range.

Meanwhile, the base may include a functional group of hydroxyl group or amine group at an end thereof. Further, the base may include at least one of potassium hydroxide, ammonium hydroxide and sodium hydroxide, or at least one of triethanolamine, aminomethyl propanol, and tromethamine.

Cosmetic products using an existing natural plant have been produced by a first method for using a distilled or extracted material obtained by heating, pressing, and distilling or extracting the plant, or a second method for containing a finely pulverized natural plant in the cosmetic products by simply performing a process of finely pulverizing the natural plant. There have been problems in case of the first method that required active ingredients are extracted in trace amounts only, and are simply used in the form of an extract only, while there has been a problem in case of the second method that it is impossible to obtain a separate visual effect or an effect of using active ingredients containing in the plant except for a fact that the natural plant is added to the cosmetic products although the cosmetic products contain the natural plant. Further, although there has been an existing method for simply performing a softening process to simply use a natural plant as a component of a cosmetic product in addition to the above-described methods, the method comprises performing a process of simply softening or pretreating the natural plant such that the natural plant is conveniently used as a cosmetic composition, but is not a method for allowing the natural plant to be removed on the skin of the user only by a finger force when a user uses a cosmetic product while intactly maintaining shape of the natural plant as in the present disclosure.

A cosmetic composition preparation method according to the present disclosure not only has high merchantability by allowing a natural plant to maintain its shape even in a cosmetic product, i.e., a final product, thereby obtaining a very excellent visual effect, but also may have very excellent efficacies of the active ingredients by enabling most of active ingredients remaining in the natural plant to be absorbed into the skin of the user since the cosmetic product component is completely disintegrated and removed when applying only a slight force to the cosmetic product component or rubbing the cosmetic product component against the skin after the user puts a cosmetic product component including a softened natural plant of which shape is remained on the user's own skin. In other words, a cosmetic composition preparation method according to the present disclosure can be said to be very meaningful by maintaining shape of a softened natural plant in a cosmetic composition prepared by a cosmetic composition preparation method according to the present disclosure and allowing almost all ingredients of the natural plant to be dissolved into the skin of a user or remained on the skin thereof during using of the cosmetic composition, thereby maximizing efficacy of the natural plant itself compared to an existing cosmetic composition preparation method for simply softening a natural plant to use a softened natural plant as one of raw materials for a cosmetic product, or for using only some ingredients of the natural plant by completely decomposing the natural plant to an extent that shape of the natural plant is removed.

On the other hand, the softening step may comprise performing a process of softening the aqueous solution in a temperature range of 40 to 95° C. for a softening time of 30 to 240 minutes.

The cosmetic composition preparation method comprises a step of softening an aqueous solution in which the natural plant is impregnated with purified water and acid in a temperature range of 40 to 95° C. for a softening time of 30 to 240 minutes. The softening step, as a nonrestrictive example, may comprise a step of softening the natural plant impregnated with the aqueous solution for the time after impregnating a natural plant with an aqueous solution in which purified water and acid that have been prepared in advance to satisfy the temperature range are mixed, or a step of softening the natural plant impregnated with the aqueous solution for the time after impregnating the natural plant with the aqueous solution and increasing temperature of the natural plant impregnated with the aqueous solution to the temperature range. Meanwhile, the softening, as another example, may be interpreted as a mean including getting soft, softened, tissues being properly disassembled, and others although the term “softening” may be used in the present specification, and the softening may include the above-mentioned meanings although it is decided to use the term “softening” for convenience in the present specification.

Since an effect of the softening process is insignificant when the aqueous solution has a temperature of less than 40° C., degree of softening may be decreased, and the natural plant may not be completely decomposed accordingly in a process of using a cosmetic composition by a user. Further, since the natural plant is completely decomposed when the aqueous solution has a temperature of more than 95° C., shape of the natural plant may not be remained. Further, usability may be lowered since the natural plant may not be completely decomposed or disintegrated when a user uses a cosmetic composition as degree of softening is insignificant when performing a softening process in the temperature range for a softening time of less than 30 minutes, while shape of the natural plant may not be remained since the natural plant is completely decomposed when performing the softening process for a softening time of more than 240 minutes. For example, the step of softening the aqueous solution may comprise performing a process of softening the aqueous solution in a temperature range of the aqueous solution of 50 to 90° C. or a temperature range of the aqueous solution of 70 to 90° C. Further, as a preferred example, the step of softening the aqueous solution may comprise performing a process of softening the aqueous solution in a temperature range of 70 to 90° C. for a softening time of 60 to 180 minutes or a softening time of 120 to 150 minutes. Further, the step of softening the aqueous solution may comprise performing a process of softening the aqueous solution in a temperature range of 75 to 85° C. for a softening time of 60 to 180 minutes or a softening time of 120 to 150 minutes. The foregoing range may enable petals to be appropriately softened, and may prevent a phenomenon that some of softened petals come loose or are disintegrated such that the softened petals disappear. Further, the foregoing range may increase a probability of softening the natural plant. That is, the foregoing range may prevent a softened natural plant from being disintegrated or decomposed, and may enable the softened natural plant to be easily decomposed (allow the softened natural plant to be absorbed into the skin or to exist on epidermis of the skin in a decomposed state by dissolving the softened natural plant on the skin of the user) when a user uses a softened natural plant.

Further, the step of softening the aqueous solution may comprise performing a process of softening the aqueous solution in a temperature range of more than 90° C. to not more than 95° C. for a softening time of 30 to 150 minutes or a softening time of 30 to 120 minutes. The foregoing range may not enable the softened natural plant to be disintegrated or decomposed, and may enable the softened natural plant to be easily decomposed when the user uses the softened natural plant.

In the case of exceeding the foregoing temperature range and softening time of the present disclosure, the user may not obtain an effect of allowing active ingredients of the natural plant to be penetrated into the skin of the user as the natural plant is completely decomposed when a user uses the natural plant while maintaining shape of a softened natural plant to be obtained in the present disclosure.

Meanwhile, a cosmetic composition preparation method according to the present disclosure may further comprise a step of confirming whether the natural plant has been softened or not as desired after the softening step. The step of confirming whether the natural plant has been softened or not, as a nonrestrictive example, may comprise, after passing through the softening step, performing a confirming process while rubbing the softened natural plant against the back of the hand by taking out a softened natural plant, putting the softened natural plant on the back of a hand, and applying a predetermined force to the softened natural plant.

Further, the step of confirming whether the natural plant has been softened or not may comprise performing a confirming process by comparing a tensile strength value of the softened natural plant with that of a dried natural plant using a tensile strength meter. As a nonrestrictive example, the tensile strength value may vary according to types of the natural plant used, and a tensile strength value of the softened natural plant may be preferably 25 to 80% of that of an initially dried natural plant.

Further, the step of confirming whether the natural plant has been softened or not may comprise performing a process of measuring any one selected from hardness, adhesiveness, springiness, cohesiveness, gumminess, chewiness, and resilience of the natural plant passing through the softening step. The hardness may be in a range of 10 to 100 g as a gravitational unit. For example, it is confirmed that the hardness becomes a range of 15 to 70 g as a gravitational unit. On the other hand, 1 g in the gravitational unit means 9.8 m/s².

Further, the adhesiveness may be in a range of −0.2 to −1.2 g·sec, and, for example, it is confirmed that the adhesiveness becomes a range of −0.3 to −1.0 g·sec.

Further, the springiness may be in a range of 0.03 to 0.22, and, for example, it is confirmed that the springiness becomes a range of 0.04 to 0.11.

Further, the cohesiveness may be in a range of 0.2 to 0.5, and, for example, it is confirmed that the cohesiveness becomes a range of 0.35 to 0.45.

Further, the gumminess may be in a range of 1 to 100, and, for example, it is confirmed that the gumminess becomes a range of 5 to 40.

Further, the chewiness may be in a range of 0.1 to 4, and, for example, it is confirmed that the chewiness becomes a range of 0.5 to 2.5.

Further, the resilience may be in a range of 0.1 to 1.8, and, for example, it is confirmed that the resilience becomes a range of 0.3 to 1.

The hardness, adhesiveness, springiness, cohesiveness, gumminess, chewiness, and resilience are measured by a texture analyzer (Model TAXT Express: Lamy rheology in France), and the measurement process is performed by using a rod having probe No. P/2Ø (diameter) 2 mm for five seconds in a state of maintaining a measurement speed of 1 mm/sec and a gap of 5 mm between a probe of the texture analyzer and a sample. Further, the gravitational unit range is an average value of result values obtained by measuring the natural plant for 10 or more times.

Meanwhile, the confirming step, as a nonrestrictive example, may comprise repeatedly performing the softening step for a softening time range of 15 to 45 minutes when the softening process is less accomplished. Namely, the confirming step may comprise repeatedly performing the softening step again when the softening process is less accomplished.

The repeatedly performing the softening step may comprise performing the softening step while additionally heating the natural plant to a temperature range of 5 to 10° C. compared to temperature of the previous softening step, and, as a nonrestrictive example, may comprise performing the softening step while reducing a softening time within a range of 5 to 10 minutes when repeatedly performing the softening step. Accordingly, degree of softening a natural plant may be adjusted more elaborately.

Next, a cosmetic composition preparation method according to the present disclosure may comprise a step of washing the natural plant softened by the softening step by using purified water. The washing step, as a step of removing a base or acid remained in the natural plant after finishing the softening process, can prevent an undesired softened natural plant from being produced as the natural plant is continuously softened by the base or acid remained in the natural plant.

Meanwhile, as a nonrestrictive example, the washing step may comprise repeatedly performing a washing process using the purified water about three to six times. The washing step may be a step of performing a process for obtaining a desired pH value of the softened natural plant.

The washing step may be performed to maintain a pH value of purified water finishing the washing process in the washing step to a range of 2.5 to 4.5. Namely, it can be said in the washing step, as a step of performing a process for removing the base or acid remained in the natural plant, that a base or acid partially remained in a softened natural plant is mixed with the purified water finishing the washing process when the base or acid is removed to some extent, and a pH value of the softened natural plant is substantially the same as that of the purified water if a pH value range of the purified water satisfies the above-mentioned pH value range. Accordingly, the washing step may be finished when the softened natural plant is in a pH value range of 2.5 to 4.5, that is, the purified water finishing the washing process is in a pH value range of 2.5 to 4.5.

Meanwhile, as a nonrestrictive example, a step of preparing the dried natural plant may further comprise a step of pulverizing the natural plant to a size of 50 to 10,000 μm. This step may be a step of appropriately performing a process of softening a natural plant in a cosmetic composition prepared through the preparation process to a desired level in the present disclosure.

Another aspect of the present disclosure provides a cosmetic composition prepared by the foregoing cosmetic composition preparation method, the cosmetic composition comprises a softened natural plant and purified water, wherein the softened natural plant may be produced by the foregoing cosmetic composition preparation method.

On the other hand, the softened natural plant and purified water may be mixed at a weight ratio of 1:1 to 1:10. The composition range can prevent a phenomenon that the softened natural plant is excessively decomposed in a process of adding a softened natural plant to a cosmetic product composition, or a phenomenon that the natural plant softened during decomposition or storage is dried and hardened again in a process of removing purified water.

Hereinafter, the present disclosure will be described in more detail with reference to specific examples. However, the following examples help understanding of the specification, and the following contents do not limit contents of the present disclosure.

### EXAMPLES 1 TO 3

After preparing rose petals as a dried natural plant to an average size of 2,000 μm, aqueous solutions containing purified water and an acidic component in the same contents as in the following Table 1 were prepared. Citric acid including a carboxyl functional group was prepared as the acidic component. Then, after impregnating the dried natural plant with the aqueous solutions, the dried natural plant was well stirred in the aqueous solutions such that the dried natural plant is completely impregnated with the aqueous solutions. After that, the aqueous solutions with which the dried natural plant had been completely impregnated were heated to 80° C. for 2 hours. After completing a softening process, filtering aqueous solutions containing an acidic component using a filter, washing and filtering filtrates three to six times using separate purified water, pH values of resulting materials were measured. A washing process was finished when pH values of washing water measured in accordance with cosmetic product formulations to be added were in a range of 2.5 to 4.5.

TABLE 1

| | | Example 1 | Example 2 | Example 3 |
|---|---|---|---|---|
| | | (wt %) | (wt %) | (wt %) |
| Components | Purified water | 98.0 | 70.0 | 55.0 |
| | (wt %) | | | |
| | Acidic | 1.0 | 15.0 | 30.0 |
| | component | | | |
| | (wt %) | | | |
| | Dried natural | 1.0 | 15.0 | 15.0 |
| | plant (wt %) | | | |

| | | | |
|---|---|---|---|
| Number of washings | 3 | 4 | 4 |
| pH | 3.25 | 3.40 | 3.20 |

FIG. 1 is a photograph showing one of the above-described cosmetic compositions of Examples 1 to 3. As shown in a cosmetic composition photograph of FIG. 1, it can be confirmed in a cosmetic composition according to the present disclosure that a softened natural plant exists while maintaining its shape within the cosmetic composition.

Meanwhile, FIG. 2 is a photograph showing a cosmetic product containing a softened natural plant prepared by using the above-described cosmetic composition. As shown in FIG. 2, it can be confirmed that the softened natural plant maintains its shape within a cosmetic product. In this way, since the softened natural plant maintains its shape within the cosmetic product, a cosmetic product according to the present disclosure can exhibit a very excellent visual effect to users of using the cosmetic product according to the present disclosure.

FIG. 3 is a photograph showing that the user uses the cosmetic product after a user directly puts the above-described cosmetic product of FIG. 2 on the back of a hand of the user.

More specifically, a photograph of using the cosmetic product in FIG. 3 is a state that the user puts the softened natural plant on the back of a hand of the user after taking out the softened natural plant within the cosmetic product along with other formulation. It can be confirmed that shape of the softened natural plant is maintained even in a state that the softened natural plant is put on the back of the hand of the user.

FIG. 4 is a photograph showing an effect that softened petals are dissolved on the skin of the user after the user uses a cosmetic product produced according to an embodiment of the present disclosure as in FIG. 3.

More specifically, it can be confirmed that all of the softened petals are finally decomposed when the user rubs the softened petals with an appropriate level of finger force in a state that softened petals are put on the back of the hand of the user. Accordingly, it can be confirmed that there is an excellent effect of enabling most of active ingredients to be utilized by allowing the main ingredients of petals to be dissolved into the skin of the user in a state that most of main ingredients of petals are not lost.

### EXAMPLES 4 TO 15

A softening process was finished after preparing calendula petals to impregnate the calendula petals with aqueous solutions containing citric acid as in an embodiment of the present disclosure, heating the calendula petals impregnated with the aqueous solutions to a temperature of 50° C., and softening the heated calendula petals impregnated with the aqueous solutions for a specific time. Specific conditions for Examples 4 to 15 are the same as in the following Table 2.

TABLE 2

| | Temperature | Softening time | Concentration of acid |
|---|---|---|---|
| | (° C.) | (min) | (wt %) |
| Example 4 | 50 | 30 | 0.5 |
| Example 5 | 50 | 30 | 1.0 |
| Example 6 | 50 | 30 | 2.0 |
| Example 7 | 50 | 60 | 0.5 |
| Example 8 | 50 | 60 | 1.0 |
| Example 9 | 50 | 60 | 2.0 |
| Example 10 | 50 | 90 | 0.5 |
| Example 11 | 50 | 90 | 1.0 |
| Example 12 | 50 | 90 | 2.0 |
| Example 13 | 50 | 120 | 0.5 |
| Example 14 | 50 | 120 | 1.0 |
| Example 15 | 50 | 120 | 2.0 |

### COMPARATIVE EXAMPLES 1 TO 4

Calendula petals were softened in a state that acid was not included in the calendula petals while setting the same temperature condition and softening time as in Examples 4 to 15. Specific conditions for Comparative Examples 1 to 4 are the same as in the following Table 3.

TABLE 3

| | Temperature | Softening time | Concentration of acid |
|---|---|---|---|
| | (° C.) | (min) | (wt %) |
| Comparative | 50 | 30 | 0.0 |
| Example 1 | | | |
| Comparative | 50 | 60 | 0.0 |
| Example 2 | | | |
| Comparative | 50 | 90 | 0.0 |
| Example 3 | | | |
| Comparative | 50 | 120 | 0.0 |
| Example 4 | | | |

Resulting products of the above-described Examples 4 to 15 and resulting products of the above-described Comparative Examples 1 to 4 are shown in photographs of FIG. 5 to FIG. 8, and conditions for the respective Examples and Comparative Examples are marked as in the photographs. For convenience of explanation, after fixing temperature and time in the resulting products of the Examples and Comparative Examples, the resulting products of the Comparative Examples are disposed on the leftmost side, and resulting products of which acid concentrations are gradually increased as it goes toward the right side are disposed from the left side in the respective photographs. Namely, the resulting products are disposed in order of Comparative Example 1, Example 4, Example 5 and Example 6 as it goes from the left side to the right side in FIG. 5, the resulting products are disposed in order of Comparative Example 2, Example 7, Example 8 and Example 9 as it goes from the left side to the right side in FIG. 6, the resulting products are disposed in order of Comparative Example 3, Example 10, Example 11 and Example 12 as it goes from the left side to the right side in FIG. 7, and the resulting products are disposed in order of Comparative Example 4, Example 13, Example 14 and Example 15 as it goes from the left side to the right side in FIG. 8.

As can be seen from FIG. 5 to FIG. 8, it can be confirmed that the acid is penetrated into calendula petals to change internal tissues of the calendula petals and change specific gravity of the calendula petals to some extent when the acid is included, and a temperature condition of the present disclosure is satisfied compared to when an acid is not included.

### EXPERIMENTAL EXAMPLES 1 TO 16

Meanwhile, after rubbing the calendula petals with an appropriate level of finger force in a state that calendula petals were put on the back of a hand of a user to measure degree of softening of calendula petals, the resulting products of the above-described Examples 4 to 15 and the resulting products of the above-described Comparative Examples 1 to 4, and observing whether the petals had been softened or not, observation results are shown in the following Table 4.

TABLE 4

| Classification | Degree of softening |
|---|---|
| Example 4 | Δ |
| Example 5 | ΔΔ |
| Example 6 | ΔΔ |
| Example 7 | Δ |
| Example 8 | ΔΔ |
| Example 9 | ΔΔ |
| Example 10 | ΔΔ |
| Example 11 | ◯ |
| Example 12 | ◯ |
| Example 13 | ΔΔ |
| Example 14 | ΔΔ |
| Example 15 | ΔΔ |
| Comparative Example 1 | X |
| Comparative Example 2 | X |
| Comparative Example 3 | X |
| Comparative Example 4 | X |

X: The calendula petals had not been softened, and residues of the calendula petals had continuously been remained on the back of the hand of the user
Δ: Although the calendula petals had been softened, the residues had not been remained on the back of the hand, but had been disappeared when the calendula petals were rubbed for a long time, or a relatively somewhat large force was applied to the calendula petals.
ΔΔ: The calendula petals had been softened, the residues had not been remained on the back of the hand, but had been disappeared although a relatively small force compared to Δ was applied to the calendula petals.
◯: The calendula petals had been softened, the residues had not been remained on the back of the hand, but had been disappeared although a relatively small force compared to ΔΔ was applied to the calendula petals.
⊚: The calendula petals had been softened very well, the residues had not been remained on the back of the hand, but had been disappeared although a relatively small force compared to ◯ was applied to the calendula petals.

As shown in Table 3 above, it can be confirmed that the calendula petals are softened although there are differences in degrees of softening in case of Examples according to the present disclosure. Meanwhile, it can be confirmed in case of Comparative Examples that the calendula petals are not softened at all, or the calendula petals get soft only, but residues of the calendula petals are remained although the calendula petals are rubbed with a strong force for a long time.

### EXAMPLES 16 TO 27

A softening process was finished after preparing calendula petals to impregnate the calendula petals with aqueous solutions containing citric acid as in an embodiment of the present disclosure, heating the calendula petals impregnated with the aqueous solutions to a temperature of 70° C., and softening the heated calendula petals impregnated with the aqueous solutions for a specific time. Specific conditions for Examples 16 to 27 are the same as in the following Table 5.

TABLE 5

| | Temperature | Softening time | Concentration of acid |
|---|---|---|---|
| | (° C.) | (min) | (wt %) |
| Example 16 | 70 | 30 | 0.5 |
| Example 17 | 70 | 30 | 1.0 |
| Example 18 | 70 | 30 | 2.0 |
| Example 19 | 70 | 60 | 0.5 |
| Example 20 | 70 | 60 | 1.0 |
| Example 21 | 70 | 60 | 2.0 |
| Example 22 | 70 | 90 | 0.5 |
| Example 23 | 70 | 90 | 1.0 |
| Example 24 | 70 | 90 | 2.0 |
| Example 25 | 70 | 120 | 0.5 |
| Example 26 | 70 | 120 | 1.0 |
| Example 27 | 70 | 120 | 2.0 |

### COMPARATIVE EXAMPLES 5 TO 8

Calendula petals were softened in a state that acid was not included in the calendula petals while setting the same temperature condition and softening time as in Examples 16 to 27. Specific conditions for Comparative Examples 5 to 8 are the same as in the following Table 6.

TABLE 6

| | Temperature | Softening time | Concentration of acid |
|---|---|---|---|
| | (° C.) | (min) | (wt %) |
| Comparative | 70 | 30 | 0.0 |
| Example 5 | | | |
| Comparative | 70 | 60 | 0.0 |
| Example 6 | | | |
| Comparative | 70 | 90 | 0.0 |
| Example 7 | | | |
| Comparative | 70 | 120 | 0.0 |
| Example 8 | | | |

Resulting products of the above-described Examples 16 to 27 and resulting products of the above-described Comparative Examples 5 to 8 are shown in photographs of FIG. 9 to FIG. 12, and conditions for the respective Examples and Comparative Examples are marked as in the photographs. For convenience of explanation, after fixing temperature and time in the resulting products of the Examples and Comparative Examples, the resulting products of the Comparative Examples are disposed on the leftmost side, and resulting products of which acid concentrations are gradually increased as it goes toward the right side are disposed from the left side in the respective photographs. Namely, the resulting products are disposed in order of Comparative Example 5, Example 16, Example 17 and Example 18 as it goes from the left side to the right side in FIG. 9, the resulting products are disposed in order of Comparative Example 6, Example 19, Example 20 and Example 21 as it goes from the left side to the right side in FIG. 10, the resulting products are disposed in order of Comparative Example 7, Example 22, Example 23 and Example 24 as it goes from the left side to the right side in FIG. 11, and the resulting products are disposed in order of Comparative Example 8, Example 25, Example 26 and Example 27 as it goes from the left side to the right side in FIG. 12.

As can be seen from FIG. 9 to FIG. 12, it can be confirmed that the acid is penetrated into calendula petals to change internal tissues of the calendula petals and change specific gravity of the calendula petals to some extent when the acid is included, and a temperature condition of the present disclosure is satisfied compared to when an acid is not included. These may be said to be similar to those of FIG. 5 to FIG. 8.

### EXPERIMENTAL EXAMPLES 17 TO 32

Meanwhile, after rubbing the calendula petals with an appropriate level of finger force in a state that calendula petals were put on the back of a hand of a user to measure degree of softening of calendula petals, the resulting products of the above-described Examples 16 to 27 and the resulting products of the above-described Comparative Examples 5 to 8, and observing whether the petals had been softened or not, observation results are shown in the following Table 7.

TABLE 7

| Classification | Degree of softening |
|---|---|
| Example 16 | Δ |
| Example 17 | Δ |
| Example 18 | ΔΔ |
| Example 19 | Δ |
| Example 20 | ΔΔ |
| Example 21 | ΔΔ |
| Example 22 | Δ |
| Example 23 | ◯ |
| Example 24 | ◯ |
| Example 25 | ΔΔ |
| Example 26 | ΔΔ |
| Example 27 | ΔΔ |
| Comparative Example 5 | X |
| Comparative Example 6 | X |
| Comparative Example 7 | X |
| Comparative Example 8 | X |

X: The calendula petals had not been softened, and residues of the calendula petals had continuously been remained on the back of the hand of the user
Δ: Although the calendula petals had been softened, the residues had not been remained on the back of the hand, but had been disappeared when the calendula petals were rubbed for a long time, or a relatively somewhat large force was applied to the calendula petals.
ΔΔ: The calendula petals had been softened, the residues had not been remained on the back of the hand, but had been disappeared although a relatively small force compared to Δ was applied to the calendula petals.
◯: The calendula petals had been softened, the residues had not been remained on the back of the hand, but had been disappeared although a relatively small force compared to ΔΔ was applied to the calendula petals.
⊚: The calendula petals had been softened very well, the residues had not been remained on the back of the hand, but had been disappeared although a relatively small force compared to ◯ was applied to the calendula petals.

As shown in Table 7 above, it can be confirmed that the calendula petals are softened although there are differences in degrees of softening in case of Examples according to the present disclosure. Meanwhile, it can be confirmed in case of Comparative Examples that the calendula petals are not softened at all, or the calendula petals get soft only, but residues of the calendula petals are remained although the calendula petals are rubbed with a strong force for a long time.

### EXAMPLES 28 TO 39

A softening process was finished after preparing calendula petals to impregnate the calendula petals with aqueous solutions containing citric acid as in an embodiment of the present disclosure, heating the calendula petals impregnated with the aqueous solutions to a temperature of 90° C., and softening the heated calendula petals impregnated with the aqueous solutions for a specific time. Specific conditions for Examples 28 to 39 are the same as in the following Table 8.

TABLE 8

| | Temperature | Softening time | Concentration of acid |
|---|---|---|---|
| | (° C.) | (min) | (wt %) |
| Example 28 | 90 | 30 | 0.5 |
| Example 29 | 90 | 30 | 1.0 |
| Example 30 | 90 | 30 | 2.0 |
| Example 31 | 90 | 60 | 0.5 |
| Example 32 | 90 | 60 | 1.0 |
| Example 33 | 90 | 60 | 2.0 |
| Example 34 | 90 | 90 | 0.5 |
| Example 35 | 90 | 90 | 1.0 |
| Example 36 | 90 | 90 | 2.0 |
| Example 37 | 90 | 120 | 0.5 |
| Example 38 | 90 | 120 | 1.0 |
| Example 39 | 90 | 120 | 2.0 |

### COMPARATIVE EXAMPLES 9 TO 12

Calendula petals were softened in a state that acid was not included in the calendula petals while setting the same temperature condition and softening time as in Examples 28 to 39. Specific conditions for Comparative Examples 9 to 12 are the same as in the following Table 9.

TABLE 9

| | Temperature | Softening time | Concentration of acid |
|---|---|---|---|
| | (° C.) | (min) | (wt %) |
| Comparative | 90 | 30 | 0.0 |
| Example 9 | | | |
| Comparative | 90 | 60 | 0.0 |
| Example 10 | | | |
| Comparative | 90 | 90 | 0.0 |
| Example 11 | | | |
| Comparative | 90 | 120 | 0.0 |
| Example 12 | | | |

Resulting products of the above-described Examples 28 to 39 and resulting products of the above-described Comparative Examples 9 to 12 are shown in photographs of FIG. 13 to FIG. 16, and conditions for the respective Examples and Comparative Examples are marked as in the photographs. For convenience of explanation, after fixing temperature and time in the resulting products of the Examples and Comparative Examples, the resulting products of the Comparative Examples are disposed on the leftmost side, and resulting products of which acid concentrations are gradually increased as it goes toward the right side are disposed from the left side in the respective photographs. Namely, the resulting products are disposed in order of Comparative Example 9, Example 28, Example 29 and Example 30 as it goes from the left side to the right side in FIG. 13, the resulting products are disposed in order of Comparative Example 10, Example 31, Example 32 and Example 33 as it goes from the left side to the right side in FIG. 14, the resulting products are disposed in order of Comparative Example 11, Example 34, Example 35 and Example 36 as it goes from the left side to the right side in FIG. 15, and the resulting products are disposed in order of Comparative Example 12, Example 37, Example 38 and Example 39 as it goes from the left side to the right side in FIG. 16.

As can be seen from FIG. 13 to FIG. 16, it can be confirmed that the acid is penetrated into calendula petals to change internal tissues of the calendula petals and change specific gravity of the calendula petals to some extent when the acid is included, and a temperature condition of the present disclosure is satisfied compared to when an acid is not included. These may be said to be similar to those of FIG. 5 to FIG. 12.

### EXPERIMENTAL EXAMPLES 33 TO 48

Meanwhile, after rubbing the calendula petals with an appropriate level of finger force in a state that calendula petals were put on the back of a hand of a user to measure degree of softening of calendula petals, the resulting products of the above-described Examples 28 to 39 and the resulting products of the above-described Comparative Examples 9 to 12, and observing whether the petals had been softened or not, observation results are shown in the following Table 10.

TABLE 10

| Classification | Degree of softening |
|---|---|
| Example 28 | ΔΔ |
| Example 29 | ◯ |
| Example 30 | ◯ |
| Example 31 | Δ |
| Example 32 | ΔΔ |
| Example 33 | ⊚ |
| Example 34 | ΔΔ |
| Example 35 | ◯ |
| Example 36 | ⊚ |
| Example 37 | ΔΔ |
| Example 38 | ◯ |
| Example 39 | ⊚ |
| Comparative Example 9 | X |
| Comparative Example 10 | X |
| Comparative Example 11 | X |
| Comparative Example 12 | X |

X: The calendula petals had not been softened, and residues of the calendula petals had continuously been remained on the back of the hand of the user
Δ: Although the calendula petals had been softened, the residues had not been remained on the back of the hand, but had been disappeared when the calendula petals were rubbed for a long time, or a relatively somewhat large force was applied to the calendula petals.
ΔΔ: The calendula petals had been softened, the residues had not been remained on the back of the hand, but had been disappeared although a relatively small force compared to Δ was applied to the calendula petals.
◯: The calendula petals had been softened, the residues had not been remained on the back of the hand, but had been disappeared although a relatively small force compared to ΔΔ was applied to the calendula petals.
⊚: The calendula petals had been softened very well, the residues had not been remained on the back of the hand, but had been disappeared although a relatively small force compared to ◯ was applied to the calendula petals.

As shown in Table 10 above, it can be confirmed that the calendula petals are softened although there are differences in degrees of softening in case of Examples according to the present disclosure. Meanwhile, it can be confirmed in case of Comparative Examples that the calendula petals are not softened at all, or the calendula petals get soft only, but residues of the calendula petals are remained although the calendula petals are rubbed with a strong force for a long time.

Although embodiments of the present disclosure have been described in detail above, the scope of the present disclosure is not limited thereto, but it will be apparent to those skilled in the art that various modifications and variations can be made within the scope of the present disclosure without departing from the technical idea of the present disclosure described in the claims.

1. A cosmetic composition preparation method comprising the steps of:
preparing a natural plant;
impregnating the natural plant with an aqueous solution comprising a base or acid and purified water; and
softening the aqueous solution while heating the aqueous solution.

2. The cosmetic composition preparation method of claim 1, wherein the acid includes a carboxyl group or a functional group of sulfonic acid.

3. The cosmetic composition preparation method of claim 1, wherein the acid includes an organic acid including at least one of citric acid, tartaric acid, lactic acid, glycolic acid, formic acid, malic acid, oxalic acid, salicylic acid, ascorbic acid, and glucono delta-lactone, or an inorganic acid including at least one of hydrochloric acid, sulfuric acid, nitric acid, phosphoric acid, and carbonic acid.

4. The cosmetic composition preparation method of claim 1, wherein the acid is in a concentration range of 0.1 to 4 wt % with respect to the total weight of the aqueous solution.

5. The cosmetic composition preparation method of claim 1, wherein the base includes a functional group of hydroxyl group or amine group at an end thereof.

6. The cosmetic composition preparation method of claim 1, wherein the base includes at least one of potassium hydroxide, ammonium hydroxide and sodium hydroxide, or at least one of triethanolamine, aminomethyl propanol, and tromethamine.

7. The cosmetic composition preparation method of claim 1, wherein the softening step comprises performing a process of softening the aqueous solution in a temperature range of 40 to 95° C. for a softening time of 30 to 240 minutes.

8. The cosmetic composition preparation method of claim 1, wherein the softening step comprises performing a process of softening the aqueous solution in a temperature range of 70 to 90° C. for a softening time of 60 to 180 minutes.

9. The cosmetic composition preparation method of claim 1, wherein the softening step comprises performing a process of softening the aqueous solution in a temperature range of more than 90° C. to not more than 95° C. for a softening time of 30 to 150 minutes.

10. The cosmetic composition preparation method of claim 1, further comprising a step of drying the natural plant before the step of impregnating the natural plant with the aqueous solution.

11. The cosmetic composition preparation method of claim 10, wherein the drying step comprises performing a process of drying the natural plant such that the natural plant after performing the drying process has a moisture content range of 5 to 15% with respect to the total weight of a dried natural plant.

12. The cosmetic composition preparation method of claim 1, wherein the step of preparing the natural plant further comprises a step of pulverizing the natural plant to a size of 50 to 10,000 μm.

13. The cosmetic composition preparation method of claim 1, further comprising a step of confirming whether the natural plant has been softened or not after the softening step, wherein the confirming step comprises repeatedly performing the softening step for a softening time range of 15 to 45 minutes when the softening process is less accomplished.

14. The cosmetic composition preparation method of claim 1, further comprising a step of washing the natural plant softened by the softening step by using purified water.

15. The cosmetic composition preparation method of claim 14, wherein the washing step comprises repeatedly performing a washing process using the purified water about three to six times.

16. The cosmetic composition preparation method of claim 14, wherein the washing step is performed to maintain a pH value of purified water finishing the washing process in the washing step to a range of 2.5 to 4.5.

17. The cosmetic composition preparation method of claim 1, wherein the natural plant includes rose, sunflower, chrysanthemum, calendula, cornflower, chamomile, morning glory, rose moss, garden balsam, cherry, green tea, hibiscus, peppermint, Asiatic pennywort, Portulaca oleracea, mugwort, Yerba Santa, Taraxacum platycarpum Dahlst., Plantago asiatica L., Huttuynia cordata, Aloe barbadensis, Opuntia humifusa, white willow, and cactusfruit.

18. A cosmetic composition comprising: a softened natural plant; and purified water,
wherein the softened natural plant is produced by the cosmetic composition preparation method of any one of claims 1 to 17.

19. The cosmetic composition of claim 18, wherein the softened natural plant and purified water are mixed at a weight ratio of 1:1 to 1:10.
